(19) United States
(12) Patent Application Publication
(10) Pub. No.: US 2025/0359601 A1
(43) Pub. Date: Nov. 27, 2025

(54) INHALER

(71) Applicant: KT&G CORPORATION, Daejeon (KR)

(72) Inventors: Yongmi JUNG, Daejeon (KR); Moonwon Kim, Daejeon (KR); Jun Won Shin, Daejeon (KR)

(73) Assignee: KT&G CORPORATION, Daejeon (KR)

(21) Appl. No.: 19/095,271

(22) Filed: Mar. 31, 2025

(30) Foreign Application Priority Data
May 22, 2024 (KR) 10-2024-0066203

(51) Int. Cl. A24F 42/60; A24F 7/00; A24F 42/20; A61M 15/00

(52) U.S. Cl. CPC A24F 42/60; A24F 7/00; A24F 42/20; A61M 15/0023; A61M 15/0025; A61M 15/0041

(57) ABSTRACT

An inhaler accommodates a capsule including a functional material and includes a housing including a first end that is open, a mouthpiece, at least a portion of which protrudes from the first end, a needle coupled to the mouthpiece and extending toward a capsule accommodated in the housing, and a retracting mechanism for inserting the mouthpiece into the housing, and the retracting mechanism changes a position of the mouthpiece between a first state in which at least a portion of the mouthpiece protrudes from the first end and a second state in which the mouthpiece is inserted into the housing.

### CROSS-REFERENCE TO RELATED APPLICATION

This application claims priority from Korean Patent Application No. 10-2024-0066203, filed on May 22, 2024, in the Korean Intellectual Property Office, the entire disclosure of which is incorporated herein by reference for all purposes.

### BACKGROUND

### 1. Field

The following embodiments relate to an inhaler.

### 2. Description of the Related Art

An inhaler may directly deliver a target material to a lung of a user. For example, patent application No. 2000-7010085 discloses a nicotine inhaler.

The above description is information the inventor(s) acquired during the course of conceiving the present disclosure, or already possessed at the time, and is not necessarily art publicly known before the present application was filed.

### SUMMARY

One or more embodiments may address at least the above problems and/or disadvantages and other disadvantages not described above. Also, the embodiments are not required to overcome the disadvantages described above, and an embodiment may not overcome any of the problems described above.

An embodiment provides an inhaler in which a capsule is easily replaced.

An embodiment provides an inhaler that stores a mouthpiece by retracting the mouthpiece when not in use.

An embodiment provides an inhaler that ensures hygiene when not in use.

An embodiment provides an inhaler that implements sufficient inhalation resistance.

An embodiment provides an inhaler that prevents unintended leakage of functional material powder.

An embodiment provides an inhaler that enhances aesthetics.

An embodiment provides an inhaler that reduces contamination of a needle.

The technical goals obtainable from the embodiments are not limited to the above-mentioned technical goals, and other unmentioned technical goals may be clearly understood from the following description by those having ordinary skill in the technical field to which the present disclosure pertains.

An inhaler according to an embodiment may accommodate a capsule including a functional material and the inhaler may include a housing including a first end that is open, a mouthpiece, at least a portion of which protrudes from the first end, a needle coupled to the mouthpiece and extending toward a capsule accommodated in the housing, and a retracting mechanism for inserting the mouthpiece into the housing, wherein the retracting mechanism may change a position of the mouthpiece between a first state in which at least a portion of the mouthpiece protrudes from the first end and a second state in which the mouthpiece is inserted into the housing.

An inhaler according to an embodiment may accommodate a capsule including a functional material and the inhaler may include a housing including a first end and a second end which are open, a mouthpiece, at least a portion of which protrudes from the first end, a holder accommodated in the housing and mounting a capsule thereon, a needle coupled to the mouthpiece and protruding toward the capsule, and a cap coupled to the second end and configured to open and close the second end, wherein the cap includes a cap body, a coupling part formed on an outside of the cap body and coupled to the housing, and a capsule mounting part on which the capsule is mounted at an end of the cap body, and the capsule mounting part protrudes from the cap body and includes a recess in a center thereof.

According to an inhaler in an embodiment, a capsule may be easily replaced.

According to an inhaler in an embodiment, a mouthpiece may be stored by retracting the mouthpiece when not in use.

According to an inhaler in an embodiment, hygiene may be ensured by protecting a mouthpiece from external contamination when not in use.

According to an inhaler in an embodiment, sufficient inhalation resistance may be implemented.

According to an inhaler in an embodiment, unintentional leakage of functional material powder may be prevented.

According to an inhaler in an embodiment, aesthetics may be enhanced.

According to an inhaler in an embodiment, contamination of a needle may be reduced.

The effects of the inhaler according to embodiments are not limited to the above-mentioned effects, and other unmentioned effects can be clearly understood from the following description by one of ordinary skill in the art.

Additional aspects of embodiments will be set forth in part in the description which follows and, in part, will be apparent from the description, or may be learned by practice of the disclosure.

### BRIEF DESCRIPTION OF THE DRAWINGS

The above and/or other aspects will be more apparent by describing certain embodiments with reference to the accompanying drawings, in which:

FIG. 1A is a perspective view of an inhaler according to an embodiment;

FIG. 1B is a perspective view of the inhaler from another angle according to an embodiment;

FIG. 1C is a cross-sectional view of the inhaler taken along line A-A of FIG. 1B;

FIG. 2 is a perspective view of an inhaler in which a mouthpiece is retracted according to an embodiment;

FIG. 3A illustrates an inhaler from which a mouthpiece is removed according to an embodiment;

FIG. 3B is a front view of an inhaler from which a housing is removed according to an embodiment;

FIG. 3C is an enlarged view of a portion B of FIG. 3B;

FIG. 3D illustrates a housing of an inhaler according to an embodiment; and

FIG. 4 is a perspective view of a cap of an inhaler according to an embodiment.

### DETAILED DESCRIPTION

The terms used in the embodiments are selected from among common terms that are currently widely used, in consideration of their function in the embodiments. However, the terms may become different according to an intention of one of ordinary skill in the art, a precedent, or the advent of new technology. Also, in particular cases, the terms are discretionally selected by the applicant of the disclosure, and the meaning of those terms will be described in detail in the corresponding part of the detailed description. Therefore, the terms used in the disclosure are not merely designations of the terms, but the terms are defined based on the meaning of the terms and content throughout the disclosure.

It will be understood that when a certain part “includes” a certain component, the part does not exclude another component but may further include another component, unless the context clearly dictates otherwise. Also, terms such as “unit,” “module,” etc., as used in the specification may refer to a part for processing at least one function or operation and may be implemented as hardware, software, or a combination of hardware and software.

As used herein, an expression such as “at least one of” that precedes listed components modifies not each of the listed components but all the components. For example, the expression “at least one of a, b, or c” should be construed as including a, b, c, a and b, a and c, b and c, or a, b, and c.

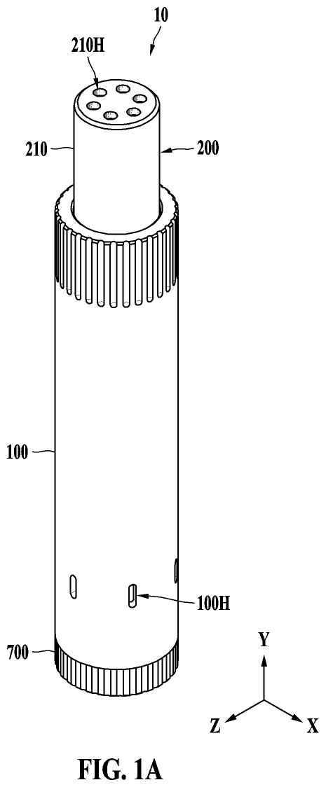

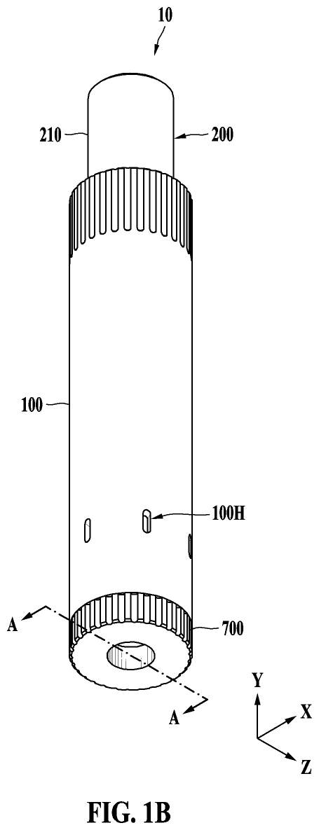

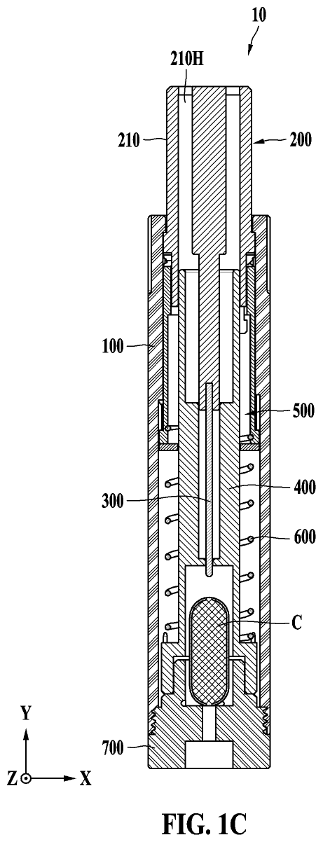

FIG. 1A is a perspective view of an inhaler according to an embodiment. FIG. 1B is a perspective view of the inhaler from another angle according to an embodiment. FIG. 1C is a cross-sectional view of the inhaler taken along line A-A of FIG. 1B.

Referring to FIGS. 1A to 1C, an inhaler 10 may deliver a functional material to a user. For example, the inhaler 10 according to an embodiment may include an inhaler that delivers nicotine to a lung of a user in the form of aerosol.

In an embodiment, the inhaler 10 may accommodate a capsule C and the capsule C may contain a functional material. For example, the functional material may include at least one of nicotine, theanine, caffeine, taurine, a pharmacological substance, or a mixture thereof. The functional material may have a form of fine granules or dry powder. For example, an outer shell of the capsule C may be composed of a material that may be penetrated or cut by a needle 300. For example, the capsule C may be filled with the functional material in an amount corresponding to 10 to 14 puffs of the user.

In an embodiment, the inhaler 10 may include a housing 100, a mouthpiece 200, the needle 300, a holder 400, a retracting mechanism 500, an elastic body 600, and a cap 700.

The housing 100 may have an internal space of which both ends are open. For example, the housing 100 may include a cylindrical shape of which both ends are open.

For example, elongated protrusion or grooves may be regularly arranged on at least a portion of an outer surface of the housing 100. The protrusions or grooves may provide a sense of grip, allowing the user to easily grip the outer surface of the housing 100.

The mouthpiece 200 may be provided at a first end (e.g., an end facing a +Y direction of FIG. 1A) of the housing. At least a portion of the mouthpiece 200 may selectively protrude from the housing 100 or may be inserted into the housing 100.

In the inhaler 10, the cap 700 may be provided at a second end (e.g., an end in a-Y direction of FIG. 1A) of the housing. The internal space of the housing 100 may be opened by the cap 700 and when opening the cap 700, the capsule C may enter the internal space of the housing 100 or may escape therefrom. The cap 700 may have a disc or cylindrical shape and may include elongated protrusions or grooves regularly formed along an outer circumferential surface. The protrusions or grooves may provide a sense of grip allowing the cap 700 to be easily open or closed.

In an embodiment, the cap 700 may include a filter. The filter may be arranged to penetrate the cap 700 in a longitudinal direction (e.g., the +Y direction of FIG. 1A) extending from the cap 700 to the mouthpiece 200. An end of the filter may be exposed to the capsule C and the other end of the filter may be exposed to external air. During a suction motion, external air may enter through the filter and the external air may facilitate the discharge of functional material powder by elevating the capsule C. For example, the filter may be a cellulose acetate filter. The filter may be a consumable. Alternatively, the filter may be used semi-permanently.

In an embodiment, the housing 100 may include a housing airflow inlet hole 100H. The external air may enter the internal space of the housing 100 through the housing airflow inlet hole 100H. The housing airflow inlet hole 100H may be formed on a side surface (e.g., a surface perpendicular to an XZ plane of FIG. 1A) of the housing 100. The housing airflow inlet hole 100H may be a hole formed by penetrating the side surface of the housing 100. For example, a plurality of housing airflow inlet holes 100H may be provided and the plurality of housing airflow inlet holes 100H may be arranged at equal intervals or equal angles along the side surface of the housing 100.

In an embodiment, the mouthpiece 200 may have a cylindrical mouthpiece body 210. The mouthpiece body 210 may move forward and backward in a longitudinal direction. The mouthpiece 200 may move between a first state in which the mouthpiece 200 protrudes from the first end of the housing 100 and a second state in which the mouthpiece 200 is inserted into the housing 100. In the first state, a portion of the mouthpiece 200 may be exposed to the outside of the housing 100 and the user may inhale the functional material by biting the exposed portion of the mouthpiece 200.

In an embodiment, the mouthpiece body 210 may have a protruding portion in a width direction (e.g., a direction parallel with an XY plane of FIG. 1A). The protruding portion in the width direction of the mouthpiece body 210 may be accommodated in the internal space of the housing 100 and may prevent the mouthpiece body 210 from escaping to the outside of the housing 100. For example, the movement of the mouthpiece body 210 may be restricted by the protruding portion in the width direction of the mouthpiece body 210.

The mouthpiece 200 may include a mouthpiece inhalation hole 210H. The mouthpiece inhalation hole 210H may be formed at an upper end (e.g., a side facing the +Y direction of FIG. 1A) of the mouthpiece body 210. The mouthpiece inhalation hole 210H may penetrate the mouthpiece body 210 in the longitudinal direction (e.g., the +Y direction of FIG. 1A). The mouthpiece inhalation hole 210H may be arranged to extend in a straight line from the capsule C. The user may inhale the functional material through the mouthpiece inhalation hole 210H from the capsule C.

In an embodiment, a plurality of mouthpiece inhalation holes 210H may be provided and the plurality of mouthpiece inhalation holes 210H may be arranged in a circumferential direction on the upper end of the mouthpiece body 210. For example, referring to FIG. 1A, the plurality of mouthpiece inhalation holes 210H may be regularly arranged at equal intervals or equal angles in the circumferential direction along the edge of the upper end of the mouthpiece body 210. The plurality of mouthpiece inhalation holes 210H may prevent empty inhalation when inhaling functional material and may provide appropriate inhalation resistance.

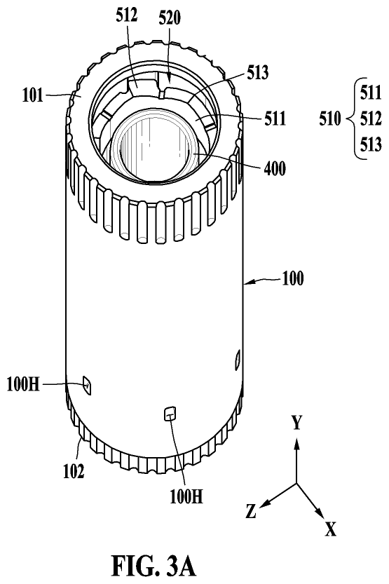

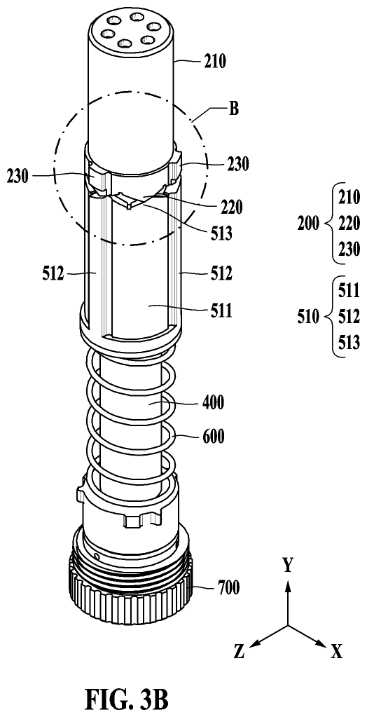

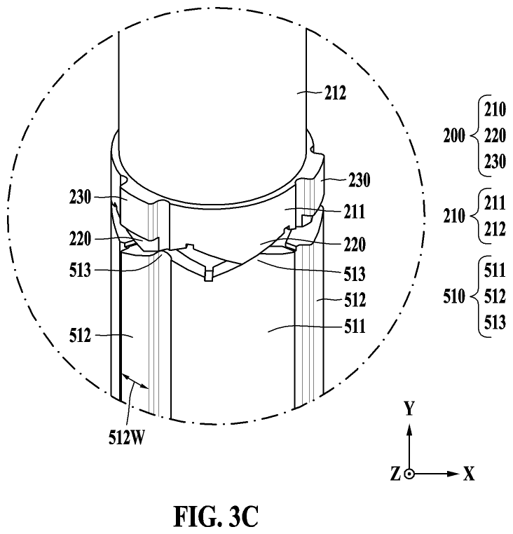

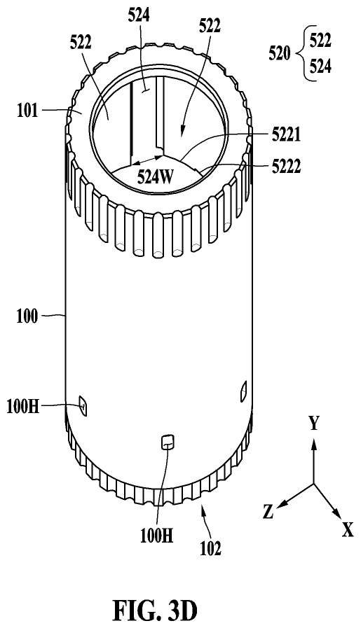

Specifically, referring to FIG. 3C, the needle 300 may be provided at a lower end (e.g., a side facing the −Y direction of FIG. 3C) of the mouthpiece body 210. The needle 300 may be provided at a position that does not overlap the mouthpiece inhalation hole 210H at the lower end of the mouthpiece body 210. For example, the needle 300 may be coupled to a center of the lower end of the mouthpiece body 210.

For example, a plurality of needles 300 may be provided. The plurality of needles 300 may be arranged at equal intervals or equal angles from the center of the lower end of the mouthpiece body 210. The plurality of needles 300 may have different lengths. For example, a center needle 300 may be the longest and an adjacent needle 300 may have shorter lengths. In this case, a central portion of the capsule C may be penetrated by the center needle 300 in the longitudinal direction and an upper portion of an outer portion of the capsule C may be cut or crushed by the adjacent needle 300. Accordingly, a vortex behavior of the airflow may be smooth in the capsule C.

The needle 300 may move together with the mouthpiece 200. For example, when the mouthpiece 200 moves forward or backward in the longitudinal direction toward the capsule C, the needle 300 may also move forward or backward in the longitudinal direction together with the mouthpiece 200. In a retracted state (e.g., the first state in which the mouthpiece 200 protrudes), the length of the needle 300 may be set so that a tip of the needle 300 is not inserted into the capsule C. When the needle 300 is in an extended state (e.g., the second state in which the user pushes the mouthpiece 200), the tip of the needle 300 may be inserted into the capsule C and at least a portion of the capsule C may be penetrated, cut, or crushed by the needle 300.

The needle 300 may not be exposed to the outside by the holder 400. Additionally, the needle 300 may not be exposed to the outside by the housing 100. Since the needle 300 is double-protected by the holder 400 and the housing 100, contamination of the needle 300 by an external environment may be prevented.

In an embodiment, the inhaler 10 may include the elastic body 600 between the mouthpiece 200 and the cap 700. More specifically, the elastic body 600 may be disposed between a sliding rotary body 510 and the cap 700. Alternatively, the elastic body 600 may be disposed between a portion of the holder 400 and the sliding rotary body 510. The elastic body 600 may provide an elastic force to push the mouthpiece 200 or the sliding rotary body 510 to their original positions when the mouthpiece 200 moves forward toward the holder 400.

The elastic body 600 may be disposed to enclose the holder 400. For example, the elastic body 600 may include a coil spring that encloses the holder 400.

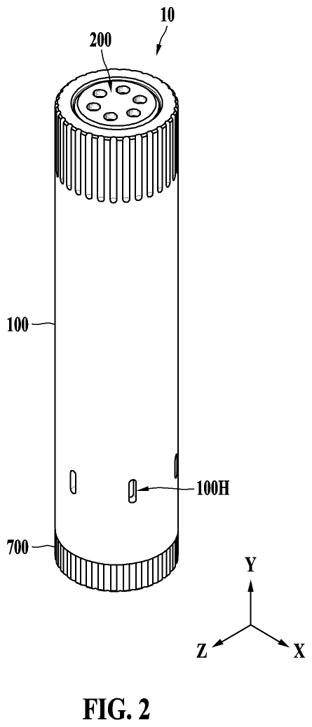

FIG. 2 illustrates a state in which the mouthpiece 200 is inserted into the housing 100. FIG. 3A illustrates the inhaler 10 from which the mouthpiece 200 is removed according to an embodiment. FIG. 3B is a front view of the inhaler 10 from which the housing 100 is removed according to an embodiment. FIG. 3C is an enlarged view of a portion B of FIG. 3B. FIG. 3D illustrates an interior of the housing 100 of the inhaler 10 according to an embodiment.

Referring to FIG. 2, the mouthpiece 200 may remain in a state (e.g., the second state) in which the mouthpiece 200 is inserted into the housing 100 when the inhaler 10 is not in use. Meanwhile, referring to FIGS. 1A, 1B, and 1C, when using the inhaler 10, the mouthpiece 200 may be in a state (e.g., the first state) in which the mouthpiece 200 protrudes from the housing 100. A retracting mechanism (e.g., the retracting mechanism 500 of FIG. 1C) may implement the second state in which the mouthpiece 200 is retracted and the first state in which the mouthpiece 200 protrudes.

FIGS. 3A to 3D illustrate the first state of the inhaler 10 and referring to FIGS. 3A to 3D, the retracting mechanism 500 may include the sliding rotary body 510 and a stopper 520.

In an embodiment, the sliding rotary body 510 may be in contact with the mouthpiece 200 and may be accommodated in the housing 100. Specifically, referring to FIGS. 3B and 3C, the sliding rotary body 510 may include a sliding rotation body 511, an engaging member 512, and a sliding rotation cam 513.

The sliding rotation body 511 may slide in the longitudinal direction in the housing 100 and may rotate in the circumferential direction of the housing 100. The sliding rotation body 511 may have a cylindrical shape of which upper and lower portions are open. The sliding rotation body 511 may be arranged concentrically with the housing 100 and/or the holder 400.

The engaging member 512 may protrude outwardly from the sliding rotation body 511. For example, the engaging member 512 may be formed to protrude lengthwise in the longitudinal direction from an outer surface of the sliding rotation body 511.

A plurality of engaging members 512 may be provided. For example, the plurality of engaging members 512 may be arranged at equal intervals or equal angles along the outer surface of the sliding rotation body 511.

The sliding rotation cam 513 may be formed at an end of the sliding rotation body 511. The sliding rotation cam 513 may face the mouthpiece 200 and may be provided at an end of the sliding rotation body 511 facing a first end 101 of the housing 100.

A plurality of sliding rotation cams 513 may be provided and the plurality of sliding rotation cams 513 may be regularly arranged at the end of the sliding rotation body 511. The sliding rotation cam 513 may be a triangular shape and may be arranged so that a corner of the triangle faces the first end 101 of the housing 100.

In an embodiment, the stopper 520 may be formed on an inner surface of the housing 100. The stopper 520 may be selectively in contact with a portion of the sliding rotary body 510.

Specifically, referring to FIG. 3D, the stopper 520 may include a engaged member 522 that protrudes toward the center of the housing 100 from the inner surface of the housing 100. A plurality of engaged members 522 may be provided and slits may be formed between the plurality of engaged members 522.

A pair of engaged elements 5221 and 5222 may be provided at an end of the engaged member 522 positioned on an opposite side of the mouthpiece 200. More specifically, the first engaged element 5221 and the second engaged element 5222 may be provided at the end of the engaged member 522 facing a second end 102 of the housing 100. The first engaged element 5221 and the second engaged element 5222 may have a triangular shape. For example, the first engaged element 5221 and the second engaged element 5222 may have a right-angled triangular shape. Due to the shapes of the first engaged element 5221 and the second engaged element 5222, a concave portion recessed toward the first end 101 of the housing 100 may be formed between the first engaged element 5221 and the second engaged element 5222. The sliding rotary body 510 may be seated on the concave portion and the inhaler 10 may remain in the second state.

A distance from a longitudinal center line of the housing 100 to the engaged member 522 may be greater than a distance from the longitudinal center line of the housing 100 to the outer surface of the sliding rotation body 511. A slit 524 may have a width 524W in the circumferential direction and the width 524W of the slit 524 may be greater than a width 512W of the engaging member 512 in the circumferential direction. By this relationship, specifically referring to FIG. 3A, when the inhaler 10 is in the first state, the engaging member 512 of the sliding rotary body 510 may be positioned in the slit 524 and the sliding rotation body 511 may be spaced apart from the engaged member 522 toward the inside of the housing 100. In this case, the longitudinal movement of the sliding rotary body 510 may not be restricted by the stopper 520.

The longitudinal movement of the sliding rotary body 510 in the first state may be restricted by the mouthpiece 200. Specifically, referring to FIG. 3C, the mouthpiece body 210 may include a first mouthpiece body 211 and a second mouthpiece body 212 in the longitudinal direction. A diameter in the width direction (e.g., a direction parallel with the XZ plane of FIG. 3C) of the first mouthpiece body 211 may be less than a diameter of an open portion at the first end 101 of the housing 100. The first mouthpiece body 211 may be an exposed portion through the first end 101 of the housing 100. The second mouthpiece body 212 may be an extended portion in the width direction compared to the first mouthpiece body 211. Since the second mouthpiece body 212 is engaged by an inwardly recessed portion from the first end 101 of the housing 100, the longitudinal movement of the second mouthpiece body 212 may be restricted. By the second mouthpiece body 212, the longitudinal movement of the mouthpiece 200 may be restricted and the mouthpiece 200 may not escape from the housing 100. Since the longitudinal movement of the mouthpiece 200 is restricted, the longitudinal movement of the sliding rotary body 510 that is in contact with the mouthpiece 200 may also be restricted.

In an embodiment, the mouthpiece 200 may further include a mouthpiece cam 220 and a guide protrusion 230.

The mouthpiece cam 220 may be provided at the end of the mouthpiece 200 facing the sliding rotation cam 513. Specifically, the mouthpiece cam 220 may be provided at an end of the second mouthpiece body 212. The mouthpiece cam 220 may have an inverted triangular shape.

Specifically, referring to FIGS. 3A to 3C, a hypotenuse of the inverted triangle of the mouthpiece cam 220 and a hypotenuse of the triangle of the sliding rotation cam 513 may be in contact with each other. As the mouthpiece body 210 moves in the longitudinal direction, the sliding rotation body 511 may be pressed in an opposite direction by the elastic body 600 and at this time, the sliding rotation body 511 may be rotated by the mouthpiece cam 220 and the sliding rotation cam 513.

In the first state, a triangular vertex of the sliding rotation cam 513 corresponding to an inverted triangular vertex of the mouthpiece cam 220 may be disposed in non-aligned positioned in the longitudinal direction to each other. Due to this positional relationship, a rotational force by the sliding rotation cam 513 may apply to the sliding rotation body 511. In this case, the rotation of the sliding rotation body 511 may be restricted by the engaging member 512 positioned in the slit 512 of the stopper 524. In addition, since the elastic body 600 pressed the sliding rotary body 510, the mouthpiece 200 may remain exposed.

The guide protrusion 230 may protrude outside of the second mouthpiece body 212. The guide protrusion 230 may be inserted into the slit 524 of the stopper 520. The guide protrusion 230 may move in the longitudinal direction along the slit 524. The mouthpiece body 210 may not rotate and move in the longitudinal direction by the guide protrusion 230.

When the user presses the mouthpiece 200 in the first state, the inhaler 10 may be switched to the second state.

With the pressing motion of the user, the mouthpiece 200 in the first state may move down toward the housing 100 and the sliding rotary body 510 may also move down together with the mouthpiece 200. In this case, since the guide protrusion 230 of the mouthpiece 200 and the engaging member 512 of the sliding rotary body 510 are accommodated in the slit 524 of the stopper 520, the sliding rotary body 510 may slide while the rotation of the sliding rotary body 510 is restricted.

As the pressing motion to the mouthpiece 200 by the user continues, the engaging member 512 of the sliding rotary body 510 may deviate from the slit 524 of the stopper 520. In this case, since the sliding rotary body 510 is freed from the engaged member 522, the rotation of the sliding rotary body 510 may occur. More specifically, first rotation of the sliding rotation body 511 may occur by the sliding rotation cam 513 of the sliding rotary body 510 and the mouthpiece cam 220 of the mouthpiece 200, and the first rotation may continue until the inverted triangular vertex of the sliding rotation cam 513 is positioned in a concave portion between adjacent sliding rotation cams 513. By the first rotation of the sliding rotation cam 513, the engaging member 512 of the sliding rotary body 10 may be disposed in a non-aligned position in the longitudinal direction relative to the slit 524 of the stopper 520.

When the pressing force is removed as the pressing motion of the user is completed, the sliding rotary body 510 and the mouthpiece 200 may move upward by the elastic body 600. In this case, the longitudinal movement of the engaging member 512 of the sliding rotary body 510 may be restricted by the engaged member 522 of the stopper 520. An inclined surface of the upper surface of the engaging member 512 of the sliding rotary body may be engaged with the second engaged element 5222 of the stopper 520. Due to the inclined surface shape of the second engaged element 5222 corresponding to the engaging member 512, the engaging member 512 may rotate in the circumferential direction while sliding in the longitudinal direction, and second rotation of the sliding rotation body 511 may occur. The second rotation may continue until the inverted triangular vertex of the sliding rotation cam 513 is positioned in a concave portion between the first engaged element 5221 and the second engaged element 5222. After the second rotation of the sliding rotation cam 513, the engaging member 512 of the sliding rotary body 510 may remain engaged with the engaged member 522 of the stopper 520, and due to this, the mouthpiece 200 may no longer be retracted along the longitudinal direction and may remain in the second state in which the mouthpiece 200 is inserted into the housing 100.

When the user presses the mouthpiece 200 again in the second state, the mouthpiece 200 and the sliding rotary body 510 may move slightly downward and the engagement between the engaging member 512 of the sliding rotary body 10 and the engaged member 522 of the stopper 520 may be released. In this case, third rotation of the sliding rotation body 511 may occur as the mouthpiece cam 220 of the mouthpiece 200 is in contact with the sliding rotation cam 513 of the sliding rotary body 510 again.

When the user releases the force to press the mouthpiece 200, the mouthpiece 200 and the sliding rotary body 510 may be retracted upward by the elastic body 600, the engaging member 512 of the sliding rotary body 510 may be in contact with the first engaged element 5221 of the stopper 520 again, and thereby, fourth rotation of the sliding rotation body 511 may occur along the inclined surface of the first engaged element 5221. The fourth rotation may continue until the engaging member 512 of the sliding rotary body 510 is inserted into the slit 524 of the stopper 520. When the engaging member 512 of the sliding rotary body 510 is inserted into the slit 524 of the stopper 520, the sliding rotation body 511 may stop rotating and retract in the longitudinal direction. The retracting of the sliding rotation body 511 may retract the mouthpiece body 210 and the mouthpiece 200 may be in the first state. In the first state, the guide protrusion 230 of the mouthpiece 200 and the engaging member 512 of the sliding rotary body 510 may be inserted into the slit 524 of the stopper 520 and may remain in an aligned state.

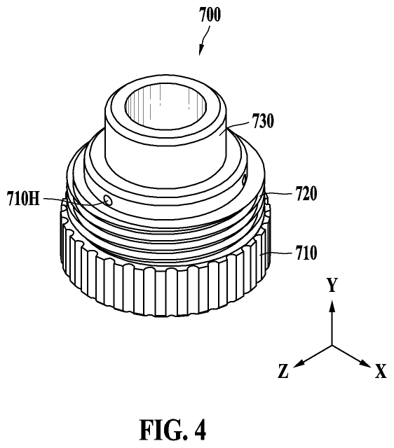

FIG. 4 illustrates a perspective view of the cap 700 of the inhaler 10 according to an embodiment.

Referring to FIG. 4, the cap 700 may be coupled to the housing 100 at a second end (e.g., the second end 102 of FIG. 3A) of the housing 100. The cap 700 may open and close the open second end 102 of the housing 100.

In an embodiment, the cap 700 may include a cap body 710, a coupling part 720, and a capsule mounting part 730. For example, the cap body 710 may have a cylindrical shape.

The coupling part 720 may include a part that is formed on the outside of the cap body 710 and is able to be coupled to the housing 100. For example, the coupling part 720 may be a screw thread formed on an outer circumferential surface of the cap body 710. A corresponding screw thread corresponding to the screw thread may be formed on the inner surface of the housing 100.

The capsule mounting part 730 may include a recess that protrudes upwardly (e.g., toward the +Y direction of FIG. 4) from the cap body 710 and accommodates the capsule C in the center thereof.

The cap 700 may be separated from the housing 100. While the capsule C is mounted on the capsule mounting part 730 of the cap 700, the cap 700 may be coupled to the housing 100. By the cap 700, the capsule C may be easily replaced and may remain in a correct position in the housing 100.

A cap airflow inlet 710H may be formed on a side surface of the cap 700. The cap airflow inlet 710H may communicate with the housing airflow inlet hole 100H. The cap airflow inlet 710H may be formed on a side surface (e.g., a surface perpendicular to the XZ plane of FIG. 4) of the cap body 710. The cap airflow inlet 710H may penetrate the cap body 710 and may extend to the capsule mounting part 730. For example, a plurality of cap airflow inlet 710H may be provided and the plurality of cap airflow inlet 710H may be arranged at equal intervals or equal angles along the cap body 710.

According to the inhaler 10 in an embodiment, the cap 700 may enable easy replacement of capsule C and may effectively prevent unintentional leakage of powder in the capsule C.

According to the inhaler 10 in an embodiment, storing the mouthpiece 200 by inserting the mouthpiece 200 into the housing 100 when not in use may protect the mouthpiece 200 from an external impact and may ensure hygiene by preventing the mouthpiece 200 from being exposed to external contamination.

According to one embodiment, the inhaler 10 may accommodate the capsule C including a functional material, and the inhaler 10 may include the housing 100 including the first end 101 that is open, the mouthpiece 200, at least a portion of which protrudes from the first end 101, the needle 300 coupled to the mouthpiece 200 and extending toward the capsule C accommodated in the housing 100, and the retracting mechanism 500 for inserting the mouthpiece 200 into the housing 100, wherein the retracting mechanism 500 may change a position of the mouthpiece 200 between a first state in which at least a portion of the mouthpiece 200 protrudes from the first end 101 and a second state in which the mouthpiece 200 is inserted into the housing 100.

In an embodiment, the retracting mechanism 500 may include the sliding rotary body 510 in contact with the mouthpiece 200 and accommodated in the housing 100, and the stopper 520 formed on an inner surface of the housing 100 and selectively in contact with a portion of the sliding rotary body 510.

In an embodiment, the sliding rotary body 510 may include the sliding rotation body 511 that is slidable in a longitudinal direction in the housing 100 and is rotatable in a circumferential direction of the housing 100, the engaging member 512 protruding outward from the sliding rotation body 511, and the sliding rotation cam 513 formed at the sliding rotation body 511 facing the mouthpiece 200.

The sliding rotation body 511 may include a cylindrical shape.

A plurality of engaging members 512 may be provided, and the plurality of engaging members 512 may be arranged at equal intervals.

The sliding rotation cam 513 may include a triangular shape, and a plurality of sliding rotation cams 513 may be provided and the plurality of sliding rotation cams 513 may be arranged regularly.

In an embodiment, the stopper 520 may include the engaged member 522 protruding from the inner surface of the housing 100, and the sliding rotation cam 513 may be selectively engaged with the engaged member 522.

A plurality of engaged members 522 may be provided, the slit 524 may be formed between the engaged members 522, and the width 524W of the slit 524 in a circumferential direction is greater than the width 512W in a circumferential direction of the engaging member 512.

An end of the engaged member 522 positioned on an opposite side of the mouthpiece 200 may include a pair of engaged elements 5221 and 5222, and the engaged elements 5221 and 5222 may include a triangular shape.

In an embodiment, the mouthpiece 200 may include the mouthpiece cam 220 having an inverted triangular shape at an end of the mouthpiece 200 facing the sliding rotation cam 513, and the mouthpiece cam 220 may be in contact with the sliding rotation cam 513.

The mouthpiece 200 may include the guide protrusion 230 protruding outward from the mouthpiece 200, and in the first state, the guide protrusion 230 may be aligned with the engaging member 512 in the longitudinal direction.

In an embodiment, the inhaler 10 may further include the elastic body 600 positioned on an opposite side of the mouthpiece 200 with the sliding rotary body 510 therebetween and in contact with the sliding rotary body 510, wherein the elastic body 600 may apply an elastic force to press the sliding rotary body 510 toward the mouthpiece 200.

In an embodiment, the inhaler 10 may further include the cap 700 coupled to the housing 100 from an opposite side of the mouthpiece 200, wherein the housing 100 may further include the second end 102 that is open on an opposite side of the first end 101, and the cap 700 may open and close the second end 102.

The housing airflow inlet hole 100H may be formed on an outer surface of the housing 100.

According to an embodiment, the inhaler 10 may accommodate the capsule C including a functional material, and the inhaler 10 may include the housing 100 including the first end 101 and the second end 102 which are open, the mouthpiece 200, at least a portion of which protrudes from the first end 101, the holder 400 accommodated in the housing 100 and mounting the capsule C, the needle 300 coupled to the mouthpiece 200 and protruding toward the capsule C, and the cap 700 coupled to the second end 102 and configured to open and close the second end 102, wherein the cap 700 may include the cap body 710, the coupling part 720 formed on an outside of the cap body 710 and coupled to the housing 100, and the capsule mounting part 730 on which the capsule C is mounted at an end of the cap body 710, wherein the capsule mounting part 730 may protrude from the cap body 710 and may include a recess in a center thereof.

The descriptions of the above-described embodiments are merely examples, and it will be understood by one of ordinary skill in the art that various changes and equivalents may be made thereto. Therefore, the scope of the disclosure should be defined by the appended claims, and all differences within the scope equivalent to those described in the claims will be construed as being included in the scope of protection defined by the claims.

What is claimed is:

1. An inhaler accommodating a capsule comprising a functional material, the inhaler comprising:
a housing comprising a first end that is open;
a mouthpiece, at least a portion of which protrudes from the first end;
a needle coupled to the mouthpiece and extending toward a capsule accommodated in the housing; and
a retracting mechanism for inserting the mouthpiece into the housing;
wherein the retracting mechanism changes a position of the mouthpiece between a first state in which at least a portion of the mouthpiece protrudes from the first end and a second state in which the mouthpiece is inserted into the housing.

2. The inhaler of claim 1, wherein the retracting mechanism comprises:
a sliding rotary body in contact with the mouthpiece and accommodated in the housing; and
a stopper formed on an inner surface of the housing and selectively in contact with a portion of the sliding rotary body.

3. The inhaler of claim 2, wherein the sliding rotary body comprises:
a sliding rotation body that is slidable in a longitudinal direction in the housing and is rotatable in a circumferential direction of the housing;
an engaging member protruding outward from the sliding rotation body; and
a sliding rotation cam formed at the sliding rotation body facing the mouthpiece.

4. The inhaler of claim 3, wherein the sliding rotation body comprises a cylindrical shape.

5. The inhaler of claim 3, wherein a plurality of engaging members is provided, and
the plurality of engaging members is arranged at equal intervals.

6. The inhaler of claim 5, wherein the sliding rotation cam comprises a triangular shape, and
a plurality of sliding rotation cams is provided and the plurality of sliding rotation cams is arranged regularly.

7. The inhaler of claim 3, wherein the stopper comprises an engaged member protruding from the inner surface of the housing, and
the sliding rotation cam is selectively engaged with the engaged member.

8. The inhaler of claim 7, wherein a plurality of engaged members is provided,
a slit is formed between the engaged members, and
a width of the slit in a circumferential direction is greater than a width in a circumferential direction of the engaging member.

9. The inhaler of claim 8, wherein an end of the engaged member positioned on an opposite side of the mouthpiece comprises a pair of engaged elements, and the engaged elements comprise a triangular shape.

10. The inhaler of claim 6, wherein the mouthpiece comprises a mouthpiece cam having an inverted triangular shape at an end of the mouthpiece facing the sliding rotation cam, and
the mouthpiece cam is in contact with the sliding rotation cam.

11. The inhaler of claim 10, wherein the mouthpiece comprises a guide protrusion protruding outward from the mouthpiece, and
in the first state, the guide protrusion is aligned with the engaging member in the longitudinal direction.

12. The inhaler of claim 2, further comprising:
an elastic body positioned on an opposite side of the mouthpiece with the sliding rotary body therebetween and in contact with the sliding rotary body,
wherein the elastic body applies an elastic force to press the sliding rotary body toward the mouthpiece.

13. The inhaler of claim 1, further comprising:
a cap coupled to the housing from an opposite side of the mouthpiece,
wherein the housing further comprises a second end that is open on an opposite side of the first end, and
the cap opens and closes the second end.

14. The inhaler of claim 1, wherein a housing airflow inlet hole is formed on an outer surface of the housing.

15. An inhaler accommodating a capsule comprising a functional material, the inhaler comprising:
a housing comprising a first end and a second end which are open;
a mouthpiece, at least a portion of which protrudes from the first end;
a holder accommodated in the housing and mounting a capsule thereon;
a needle coupled to the mouthpiece and protruding toward the capsule; and
a cap coupled to the second end and configured to open and close the second end;
wherein the cap comprises:
a cap body;
a coupling part formed on an outside of the cap body and coupled to the housing; and
a capsule mounting part on which the capsule is mounted at an end of the cap body,
wherein the capsule mounting part protrudes from the cap body and comprises a recess in a center thereof.
